(19) United States
(12) Patent Application Publication
(10) Pub. No.: US 2015/0343103 A1
(43) Pub. Date: Dec. 3, 2015

(54) UV CHAMBER AND METHOD OF SANITIZING AGRICULTURAL FACILITIES USING THE SAME

(71) Applicant: Once Innovations, Inc., Plymouth, MN (US)

(72) Inventors: Zdenko Grajcar, Orono, MN (US); Brad Freking, Alpha, MN (US); Leif Erickson, Saint Paul, MN (US)

(21) Appl. No.: 14/728,261

(22) Filed: Jun. 2, 2015

Related U.S. Application Data
(60) Provisional application No. 62/006,483, filed on Jun. 2, 2014.

(51) Int. Cl. A61L 2/10; A61L 2/24

(52) U.S. Cl. CPC A61L 2/10; A61L 2/24

(57) ABSTRACT

A UV chamber for inactivating microorganisms in an agricultural facility and method of enhancing sanitation in an agricultural facility using the same. Items are placed inside the UV chamber when entering an agricultural facility. The chamber locks enclosing the item therein and a lighting device directs UV radiation toward the item for a predetermined amount of time necessary to inactivate any microorganisms on the items. The inactive microorganisms are then exposed to the animals within the facility so the animals can build immunity to the microorganism.

### CLAIM OF PRIORITY

This patent application claims the benefit of priority to U.S. Provisional Patent Application Ser. No. 62/006,483, filed on Jun. 2, 2014, entitled “UV Chamber and Method of Sanitizing Agricultural Facilities Using the Same” the entire contents of which are incorporated herein by reference.

### BACKGROUND

This invention relates to sanitation in agricultural facilities. More specifically this invention relates to the use of UV radiation to prevent the spread of disease in agricultural environments.

In the swine industry the porcine epidemic diarrhea virus (PEDV) has been identified in the U.S. for the first time. While PEDV is a virus that has existed in other parts of the world for many years, for the first time it is hitting U.S. farms. The virus is not only deadly for piglets, but extremely contagious. At this time there is not a known cure for the virus and once in a swine facility entire liters are wiped out.

As a result of PEDV the swine industry has attempted to increase sanitation measures to ensure the virus does not get into additional swine facilities. Measures include having individuals visiting swine facilities to be tested by a doctor before entry into the farm is allowed, the wash down of individuals and changing of clothes before entering a swine facility and mandatory waiting periods before a visitor is allowed to visit a swine facility. Specifically, once a visitor has been to another swine facility, at least two or even three weeks must pass before that individual is allowed back into a different swine facility.

Still besides these precautions PEDV continues to spread across the U.S. In particular, in the industry, individuals that provide feed, water and equipment must travel from farm to farm. Similarly veterinarians similarly must travel to and from different facilities. Other times individuals are not truthful about the last time they were in a different swine facility. Over time, despite all of the precautions, the virus still finds human carriers that take the disease from one swine facility to the next.

### Overview

Thus, a need in the art exists for a manner in which swine facilities can be protected against PEDV and other dangerous pathogens and microorganisms that can be detrimental to a swine facility. Specifically additional sanitation measures are required within the industry to combat disease. Therefore a principle object of the present invention is to provide an ultraviolet (UV) light chamber that inactivates viruses and other germs that are harmful with agricultural facilities.

The present inventors have recognized, among other things, that a UV chamber for inactivating microorganisms within an agricultural facility and a method of enhancing sanitation with the agricultural facility using the same can address these and other problems. A UV chamber includes an interior chamber with a tray member that receives items. Upon placement of the items within the interior of the UV chamber the interior is enclosed and sealed through a magnetic interlock. A timing device is actuated and UV radiation from at least one lighting device is directed toward the item for a predetermined amount of time to inactivate a microorganism. The inactive microorganism is then exposed to the rest of the facility where livestock is exposed to the inactive microorganism to develop an immunity to the microorganism.

This overview is intended to provide an overview of subject matter of the present patent application. It is not intended to provide an exclusive or exhaustive explanation of the invention. The detailed description is included to provide further information about the present patent application.

### BRIEF SUMMARY OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an open UV chamber.

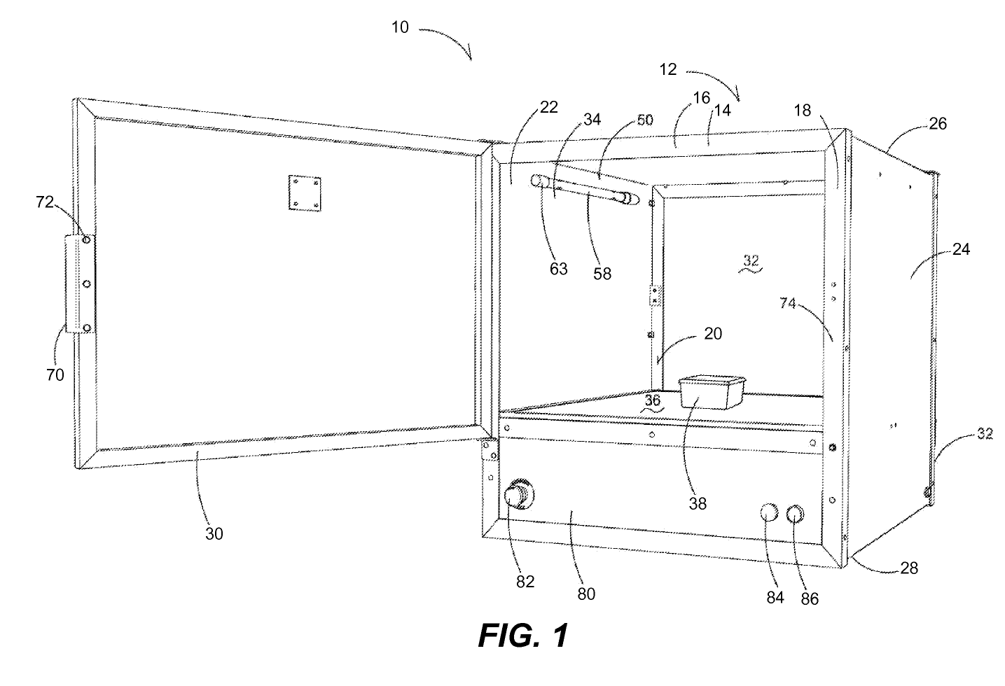

FIG. 2 is a perspective view of an enclosed UV chamber.

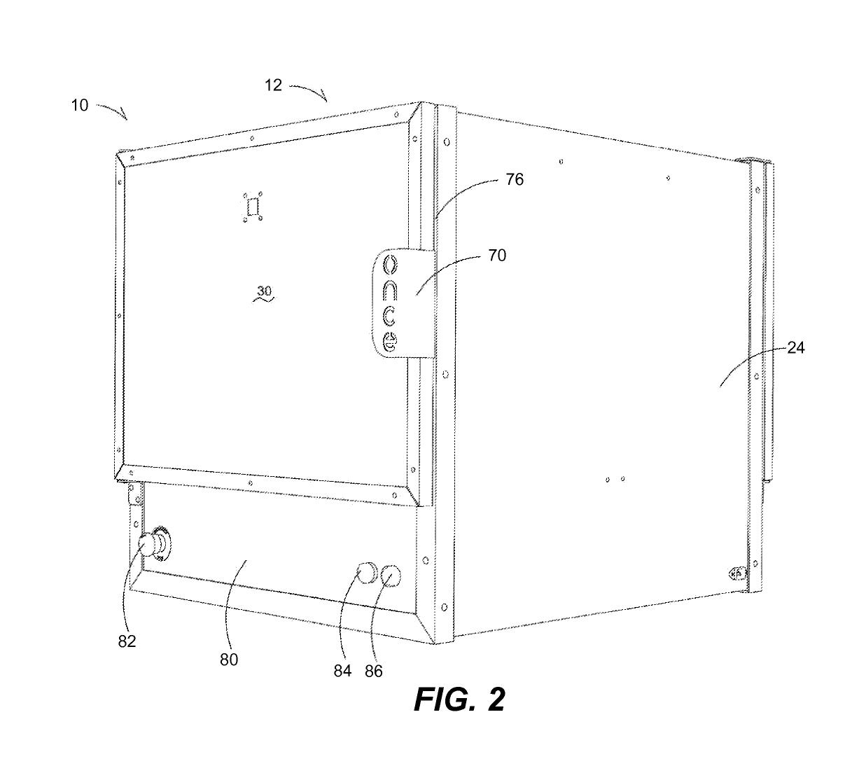

FIG. 3 is a perspective cut away view of an interior of a UV chamber.

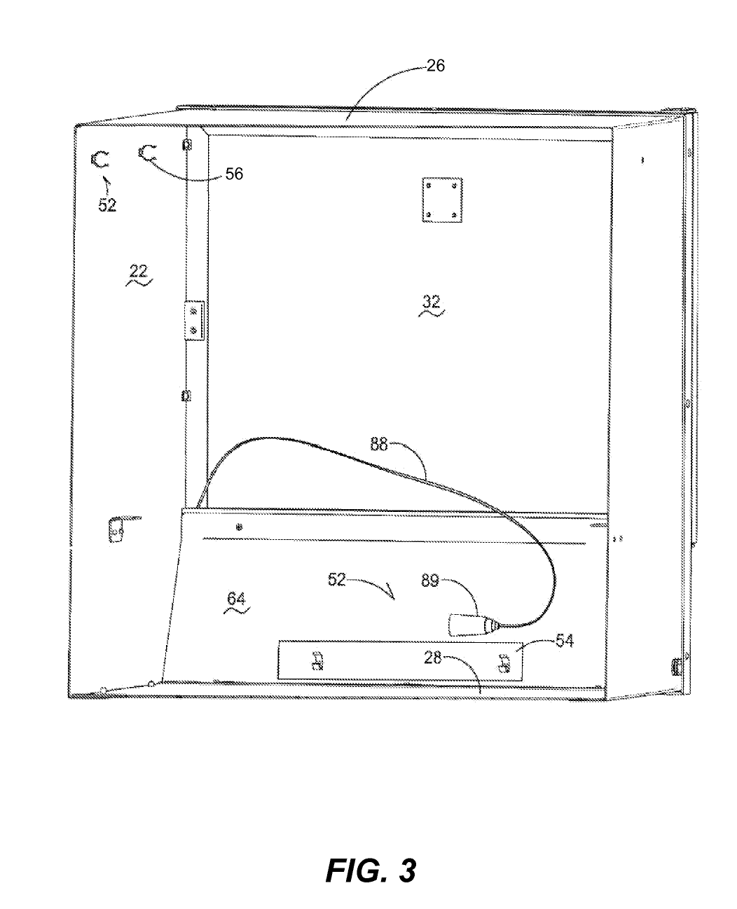

FIG. 4 is a schematic diagram of the electrical components of a UV chamber.

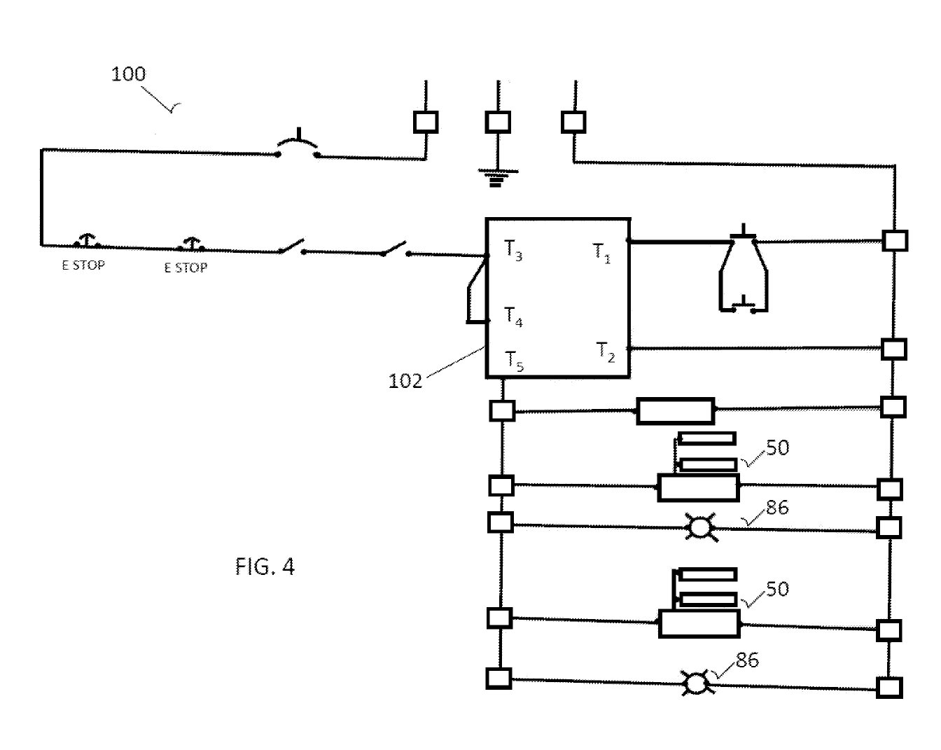

### DETAILED DESCRIPTION

The figures show a UV chamber 10 having a body 12 that in one embodiment is generally shaped like a box, or has a rectangular cross section. In particular in this embodiment the body 12 has a frame 14 with first support members 16 running in parallel spaced relation in an x axis, second support members 18 running in parallel spaced relation in a y axis and perpendicular to the first support members and third support members 20 running in parallel spaced relation in a Z axis perpendicular to both first and second support members 16 and 18, thus forming the frame 14 having a square or rectangular cross section.

The first, second and third support members 16, 18 and 20 support first and second sidewalls 22 and 24 that are in parallel spaced relation that extend between a top wall 26 and bottom wall 28. First and second door members 30 and 32 are hindgedly connected to the first and second sidewalls 22 and 24. The door members 30 and 32 while described as hindgedly connected can be connected to the first and second sidewalls 22 and 24 in other manners that allow the door members 30 and 32 to be opened to gain access to the open interior 34 of the UV chamber. This includes being connected to the top or bottom walls 26 and 28.

The interior 34 of the UV chamber includes a retractable tray member 36 on which items 38 such as clothing, pieces of jewelry, feed, packages, water containers, beverage containers and the like can be placed. In one embodiment the tray member 36 is within tracks and slide outwardly until engaging a stop member. In other embodiments the tray member 36 is on rollers and again engages a stop member. By having two separate door members 30 and 32 in association with the tray member 36 an individual can place the items 38 into the first door member 30 and then go through decontamination themselves and get their items 38 through the opposite door member 32. This provides a user with additional flexibility and functionality regarding where to locate the chamber 10.

A plurality of lighting devices 50 are also disposed within the interior 34 of the UV chamber 10. The lighting devices 50 in one embodiment are received by light holders 52 having a reflective plate 54 that preferably is made of or coated with a UV reflective material. The reflective plate 54 surrounds attachment members 56 such as c-shaped clamps. In this preferred embodiment the lighting device has a transparent tubular body 58 that preferably is made of a material that absorbs a minimal amount of UV wavelength light as the radiation/light passes through the material. Within the tubular transparent material is a printed circuit board (PCB) disposed therein that has a plurality of light emitting diodes that emit UV wavelength light, or a wavelength between 100 nm-400 nm, more preferably 200-300 nm, even more preferably between 250 nm-260 nm and more preferably approximately 254 nm, to the interior 34 of the UV chamber 10.

In this embodiment a first light holder 52 is secured to the body 12 of the UV chamber running along the corner of the UV chamber 10 created between the first sidewall 22 and the top wall 26 and extending the length of the interior 34. When in place the PCB is angled at the center of tray member 36 and the reflective plate 54 arcuately extends around the tube in approximately a half circle such that a maximum amount of light is directed directly at the tray member 36. Similarly a second light holder 52 extends in the opposite corner of the UV chamber 10 adjacent the second sidewall 24 and top wall 26 again having the same lighting device 50 in the same light holder 52 and with the same directional lighting to focus the UV radiation toward the tray member 36. While these lighting devices have been described as on a light holder 52, the lighting devices could be secured directly to the chamber 10 or on an elongated angled member built into the interior 34 of the chamber 10 or other similar mounting means without falling outside the scope of this disclosure. Also, while the lighting device 50 has been described as using LEDs as a light or radiation source, any light source, incandescent, compact fluorescent, high pressure sodium, halogen, DC driven LED, AC driven LED or the like that can emit wavelengths of light in the 200 nm-300 nm range is contemplated and fall within the scope of this disclosure.

In addition to the lighting devices 50 running along the top wall 26 of the interior 34, additional auxiliary lighting devices 50 are below the tray along each of the first and second sidewalls 22 and 24 and the bottom wall 28. Each lighting device 50 optionally can be in a light holder 52 with the reflective plate 54, attachment members 56, transparent tubular body 58, printed circuit board (PCB) and plurality of light emitting diodes that emit UV wavelength as described above angled toward the tray member 36. The lighting device 50 in one embodiment has an electrical connector 63 that can only be inserted our actuated with a predetermined connector. In this manner, if an individual were to remove the UV lighting device 50 from the chamber, they would be unable to power the lighting device 50 from a common power source or common means to ensure UV light is not emitted outside the interior 34 of the UV chamber 10. In one embodiment an angled surface 64 can extend the length of the interior again providing a directionally supported lighting device 50 where light is directly aimed at the tray member 36.

The angled surface 64 along with all the surfaces within the interior 34 of the UV chamber, including those interior surfaces of the sidewalls, 22 and 24, the top and bottom walls 26 and 28 and door members 30 and 32 are either made from, is anodized or coated with UV reflective material that absorbs a minimal amount of UV radiation. In this manner, as a result of all lighting devices 50 being directional and pointing directly at the centrally located tray member 36 and all interior surfaces having UV reflective materials the UV chamber maximizes the effect of UV radiation used to radiate any item 38 on the tray member 36. Thus the least amount of energy required is used and the effectiveness of the UV radiation is maximized.

Exterior to the chamber 10 a handle 70 is secured to each door member 30 and 32 and has a magnetic interlock 72 therein that engages a sidewall 22 or 24 or top or bottom wall 26 or 28 to provide an interlocking connection. Surrounding the exterior surface 74 of each sidewall 22 and 24 and top and bottom walls 26 and 28 is a seal 76 preferably made of elastic material and is of size and shape that upon closing of a door member 30 or 32 and the magnetic interlock 72 engages and locks the seal prevents light from lighting devices 50 from escaping the interior 34 of the chamber.

A control panel 80 is disposed below each door member 30 and 32 and includes an emergency stop button 82, a timer button 84 and an indicator light 86. Disposed within and originating from a compartment within the UV chamber 10 is electrical wiring 88 for the electronic components 50, 72, 82, 84 and 86 of the chamber 10. In one embodiment the wiring terminates in a connector 89 for the UV lighting devices 50 that only plug into the connector of the UV lighting device 50.

The figures provide a schematic diagram of the electrical system 100 of the UV chamber 10. The electrical system has an input line L, neutral line N and ground G. A circuit breaker CB1 is within in the input line L in series with first and second emergency stop switches ES1 and ES2 actuated by the emergency stop buttons 82 of each control panel 80. Control relays CR1 and first and second switches SW1 and SW2, that in one embodiment are Reed switches, provide electrical pathways to ensure that should a door member 30 or 32 be opened current no longer flows to the lighting devices 50 to ensure UV light that can be harmful to the eyes of humans cannot be emitted by the lighting devices 50 when a door member 30 or 32 is open.

A timing circuit 102 is connected to the input line L and neutral line N and is electrically connected to the timer button 84, such that when both door members 30 and 32 are closed and interlocked and a timer button 84 (PB1 and PB2) is actuated the timing circuit 102 is actuated. The timing circuit provides current to the ballasts (BL1 and BL2, and BAL1 and BAL2) of lighting devices 50 for a predetermined period of time. In one embodiment the predetermined period is approximately two minutes. The predetermined amount of time can be several seconds to several days without falling outside the scope of this disclosure.

The timing circuit 102 is also connected in parallel with the ballast (BL1 and BL2, and BALI and BAL2) for the lighting devices 50 indicator lights 86 (LT1 and LT2) such that while the lighting devices 50 receive current the indicator lights 86 similarly receive current and lights to provide a warning to users that the UV lights are emitting radiation in the chamber 10. This provides a warning to a user that the door members 30 and 32 should remain closed.

In operation, the UV chamber 10 is placed at the entrance of a swine facility. When an individual comes to the swine facility, before entering the individual takes any packages, feed, water, jewelry, shoes, clothing or the like and takes such items 38 and places them on the tray member 36. At this time the individual closes the UV chamber 10, ensuring the door members 30 and 32 are magnetically interlocked. Then the individual engages a timer button 84. If both door members 30 and 32 are not interlocked the electrical system 100 will not actuate as a result of the switches SW1 and SW2 and circuit relays C1 and the lighting devices 50 do not emit UV light that could escape the chamber and be harmful to a user.

If the door members 30 and 32 are completely closed and interlocking has occurred, the switches SW1 and SW2 and circuit relays CR1 provide a completed circuit allowing current to flow to the timing circuit 102. Thus, when the timer button 84 is actuated current flows to the ballasts (BL1 and BL2, and BALI and BAL2) of lighting devices 50 to direct UV radiation directly at the item or items 38 on the tray member 36 for a predetermined amount of time. During this time as current is flowing to the ballasts (BL1 and BL2, and BALI and BAL2) of lighting devices 50, current is simultaneously flowing to indicator lights 86 (LT1 and LT2), thus actuating the indicator lights 86 to alert a user that UV radiation is present and the door members 30 should remain closed. In one embodiment the magnetic interlocking prevents opening of the door members 30 and 32 during the predetermined period of time. If a user ignores the indicator lights 86 and is able to open a door member 30 or 32, the circuit is no longer operable as a result of the switches SW1 or SW2 and the circuit relays CR1 thus stopping current flow to the ballasts (BL1 and BL2, and BALI and BAL2) of lighting devices 50 and the indicator lights 86 shut off. If a malfunction occurs and current continues to flow to the ballasts (BL1 and BL2, and BAL1 and BAL2) of lighting devices 50 and the indicator lights 86 remain on, a user recognizing this can actuate an emergency stop switch (ES1 or ES2) by actuating the emergency stop button 82. Thus multiple methods are presented to minimize and eliminate an individual from being exposed to potentially harmful UV radiation of the lighting devices 50.

If the UV chamber 10 is operated properly and a door member 30 or 32 is not opened during the predetermined amount of time, the UV radiation radiates the item or items 38. Thus if a germ, microorganism, virus or the like is on the item 38 it is exposed to the UV radiation for the predetermined period of time.

During this period, the UV radiation is absorbed by DNA of these microorganisms breaking the molecular bonds within the micro-organismal DNA, producing thymine dimers in the DNA preventing the DNA from replicating. In this manner, cells and viruses become inactive and unable to reproduce. Thus, if a virus such as the porcine epidemic diarrhea virus is on an item 38 the exposure to the UV radiation for the predetermined amount of time inactivates the virus, preventing it from replicating. As a result the exposure of swine in a facility to only an inactive virus allows the swine to be able to be resistant and fight off the virus, and even build proteins and immunities to the virus. In this manner the inactive virus and thus the UV chamber presents and manner in which one can provide immunization for the swine within in the facility in case the swine where exposed to the virus at a later time.

In particular, the entire UVC range of ultraviolet radiation is germicidal. Ultraviolet Germicidal Irradiation (UVGI) deactivates the DNA of bacteria, viruses and all pathogens and consequently destroys their ability to multiply and cause disease. UVGI causes damage to the nucleic acid of microorganisms by forming covalent bonds between certain adjacent bases in the DNA. The formation of such bonds prevent the DNA from being unzipped for replication, and the organism is unable to reproduce. In fact, when the organism tries to replicate, it dies.

While described as combating the porcine epidemic diarrhea virus in a swine facility, the UV chamber can be used as a sanitation device in any agricultural facility, including for chickens, turkeys, cows, horses and the like and to inactivate or kill any germ, microorganism, virus or the like, both without falling outside the scope of this disclosure. In particular, the chamber 10 provides enhanced sanitation of items 38 entering an agricultural facility.

Thus provided is a UV chamber 10 that provides a manner of inactivating viruses, germs and other microorganisms through use of UV radiation to ensure an animal can fight off and become immune to the effects of such virus, germs or microorganisms. The chamber 10 also presents multiple safety features to ensure that a user is not exposed to the UV radiation that can have harmful effects on humans. Thus, at the very least, all of the stated objectives have been met.

What is claimed:

1. A method of sanitizing an agricultural facility, steps comprising:
placing a UV chamber having an open interior within the agricultural facility;
placing an item within the UV chamber;
enclosing the open interior;
directing UV radiation from at least one lighting device toward the item for a predetermined amount of time to inactivate a microorganism.

2. The method of claim 1 wherein the open interior is enclosed by a magnetically interlocked door.

3. The method of claim 1 wherein the item is placed on a tray member.

4. The method of claim 1 wherein the UV radiation is directed toward the item upon actuation of a timer button.

5. The method of claim 1 wherein upon actuation of the timer button current flows both to the lighting device and an indicator light.

6. The method of claim 1 wherein the microorganism is a porcine epidemic diarrhea virus.

7. The method of claim 1 wherein the UV radiation is between 200 nm-300 nm.

8. The method of claim 1 further comprising the step of exposing the agricultural facility to the inactivated microorganism.

9. The method of claim 1 wherein the agricultural facility is a swine facility.
